(19) United States
(12) Patent Application Publication
(10) Pub. No.: US 2024/0408267 A1
(43) Pub. Date: Dec. 12, 2024

(54) COMPOSITIONS OF DECIDUA TISSUE AND USES THEREOF

(71) Applicant: OSIRIS THERAPEUTICS, INC., Columbia, MD (US)

(72) Inventors: Nasreen JACOBSON, Columbia, MD (US); Jin-Qiang KUANG, Columbia, MD (US); Min Sung PARK, Columbia, MD (US); Aleksa JOVANOVIC, Columbia, MD (US)

(73) Assignee: OSIRIS THERAPEUTICS, INC., Columbia, MD (US)

(21) Appl. No.: 18/696,785

(22) PCT Filed: Sep. 29, 2022
(86) PCT No.: PCT/IB2022/059310

§ 371 Date: Mar. 28, 2024

Related U.S. Application Data
(60) Provisional application No. 63/249,655, filed on Sep. 29, 2021.

(51) Int. Cl. A61L 26/00; A61K 35/50

(52) U.S. Cl. CPC A61L 26/0066; A61K 35/50; A61L 26/0004; A61L 26/0019; A61L 26/0023; A61L 26/0057; A61L 26/0071; A61L 26/008; A61L 26/009; A61L 2300/412; A61L 2430/34

(57) ABSTRACT

Disclosed are compositions and methods for treating wounds and damaged tissue using decidua tissue.

### CROSS-REFERENCE WITH RELATED APPLICATIONS

This application claims the benefit of U.S. Provisional Application No. 63/249,655 filed Sep. 29, 2021, the contents of which are incorporated into the present application by reference.

### FIELD OF THE INVENTION

The present invention generally relates to compositions comprising decidua tissue, preferably dehydrated decidua tissue, and its use in the field of wound management and tissue repair.

### BACKGROUND OF THE INVENTION

A wound is a disruption of the structure and function of tissue. A dermal wound involves the disruption of the skin and associated soft tissue architecture. Dermal wounds can be partial or full thickness wounds. They can also be acute wounds, chronic wounds, or burns (which can be acute or chronic). To ensure proper healing, the wound bed should be free of devitalized tissue, be well vascularized, and moist. Wound dressings can help facilitate the wound healing process if they provide an environment to support the healing process by allowing cellular migration, vascular ingrowth, and/or the formation of granulation tissues. Many of the commercially available wound dressings do not have these capabilities to support the healing process, and some are not very effective at this. Additionally, some of these wound dressings are not cost-efficient or manageable for the medical practitioner.

### SUMMARY OF THE INVENTION

The present disclosure provides a solution to at least one or more of the aforementioned limitations and deficiencies in the art generally relating to wound management and tissue repair. The solution is premised on the use of compositions comprising decidua tissue as wound dressings.

The decidua is a mucosal tissue of the uterus that forms in preparation for pregnancy. It includes decidua basalis, decidua capsularis, and decidua parietalis. The decidua basalis is the maternal part of the placenta. The decidua is generally discarded even if the remaining placental tissue is to be processed into placental products. The inventors of the present application have surprisingly discovered that compositions of decidua tissue provide an unexpectedly greater beneficial environment for cellular attachment and proliferation of mesenchymal cells in culture as compared to many commercially available wound dressing products. Thus, the decidua tissue compositions of the present invention provide superior conditions to support the wound healing process versus existing wound dressing products. Notably, the decidua has long been considered a disposable material, which allows one to obtain decidua at little or no cost. Thus, using decidua tissue as a raw material for the compositions of the present invention also provide a cost-effective product, which can help lower health care costs associated with wound management and tissue repair.

In one aspect of the present invention, disclosed is a composition comprising decidua tissue. In some embodiments, the decidua tissue is dehydrated decidua tissue. In some embodiments, the decidua tissue or dehydrated decidua tissue comprises decidua basalis tissue, decidua capsularis tissue, or decidua parictalis tissue, or any combinations thereof. In some embodiments, the decidua tissue or dehydrated decidua tissue is non-viable. In some embodiments, the non-viable decidua tissue or dehydrated decidua tissue is rendered non-viable by cell lysis. In some embodiments, the non-viable decidua tissue or dehydrated decidua tissue is rendered non-viable by sterilization. The dehydrated decidua tissue can be obtained by lyophilizing (freeze-drying), evaporative air drying (e.g., thermal drying with heated air under gravity convection or forced air convection conditions; or drying under ambient or room temperature conditions under gravity convection or forced air convection conditions), indirect drying (e.g., vacuum drying), contact drying (e.g., drying through contact with a heated material or source), dielectric drying (e.g., use of radiofrequency or microwaves), infrared drying, and/or supercritical drying. In some embodiments, the dehydrated decidua is dehydrated by lyophilization. In some embodiments the dehydrated decidua tissue is a powder. In some embodiments, the dehydrated decidua tissue has a water content of less than 5% w/w, or less than 3% w/w. In some embodiments the composition further comprises a carrier. In some embodiments, the carrier is a pharmaceutically acceptable carrier. In some embodiments, the carrier or pharmaceutically acceptable carrier is aqueous based. In some embodiments, the aqueous based carrier comprised at least 50% w/w water. In some embodiments, the pharmaceutically acceptable carrier comprises one or more cellulose ethers. In some embodiments, the cellulose ether is a non-ionic cellulose ether. In some embodiments, the non-ionic cellulose ether is selected from hydroxyethyl cellulose (HEC) or hydroxypropyl cellulose (HPC), or mixtures thereof. In some embodiments, the pharmaceutically acceptable carrier further comprises one or more plasticizers. In some embodiments, the plasticizer is a hydrophilic polyol. In some embodiments, the hydrophilic polyol is a hydrophilic polymeric polyol. In some embodiments, the hydrophilic polymeric polyol is one or more polyethylene glycols. In some embodiments, the one or more polyethylene glycols comprises polyethylene glycol 600. In some embodiments the composition is dehydrated. In some embodiments, the composition is dehydrated by evaporative air-drying. In some embodiments, the evaporative air-drying is thermal evaporative air-drying with heated air under gravity convection or forced air convection conditions. In other embodiments, the evaporative air-drying is ambient or room temperature evaporative air-drying under gravity convection or forced air convection conditions. In still other embodiments, the evaporative air-drying is room temperature evaporative air-drying under gravity convection conditions. In other embodiments, the composition is dehydrated by lyophilization. In some embodiments, the dehydrated composition is a sheet or film. In some embodiments, the composition comprising the decidua tissue is a powder. In some embodiments, the water content of the dehydrated composition is less than 5% w/w, or less than 3% w/w. In some embodiments, the sheet or film has a thickness of from about 0.1 mm to about 25 mm, or from about 0.5 to about 10 mm, or from about 1 mm to about 5 mm. In some embodiments, the concentration of decidua tissue is from about 1% to about 75% w/w, or from about 4% to about 50% w/w of the dehydrated composition. In some embodiments, the dehydrated composition is capable of forming a hydrogel when the dehydrated composition is contacted with water or other aqueous medium. In some embodiments, the dehydrated composition is opaque. In other embodiments, the dehydrated composition is transparent. In some embodiments, the dehydrated composition is translucent. In some embodiments, the dehydrated composition comprises markings, symbols, letters, or numbers, which can be helpful in identifying the source of the composition and/or can be helpful in orienting the composition during use or application to a wound (e.g., top side opposite the wound and bottom side in contact with the wound).

In another aspect of the present invention, disclosed is a method of treating a wound in a subject, the method comprising applying the compositions of the present invention to the wound (e.g., a composition or dehydrated composition comprising decidua tissue, preferably dehydrated decidua tissue). In some embodiments, the composition is applied in conjunction with applying another wound dressing. In some embodiments, the composition or dehydrated composition is a powder. In some embodiments, the wound is a dermal wound. In some embodiments, the dermal wound is a partial thickness wound. In other embodiments, the dermal wound is a full thickness wound. In some embodiments, the dermal wound is a burn, a chronic wound, or an acute wound. In some embodiments, the dermal wound is a burn and is selected from a superficial (first degree) burn, a partial thickness (second degree) burn, a full thickness (third degree) burn, or a radiation burn. In other embodiments, the dermal wound is a chronic wound and is selected from a dermal ulcer, a diabetic ulcer, a diabetic foot ulcer, a venous ulcer, a venous leg ulcer, an arterial ulcer, an arterial leg ulcer, a decubitus ulcer, a stasis ulcer, an ischemic ulcer, a vascular ulcer, a pressure ulcer (stage I-IV), a podiatric wound, a draining wound, a tunneling wound, or an undermining wound. In still other embodiments, the dermal wound is an acute wound and is selected from a trauma wound, a laceration, an abrasion, a skin tear, a skin lesion, a blister, a surgical incision, a donor skin site, a skin graft, a laser surgery wound, a Mohs surgery wound, or a dehisced wound.

In another aspect of the present invention, disclosed is a method of treating a wound in a subject, the method comprising applying a composition of the present invention to the wound (e.g., a composition or dehydrated composition comprising decidua tissue, preferably dehydrated decidua tissue).

In another aspect of the present invention, disclosed is a method of making a dehydrated composition comprising dehydrated decidua tissue, the method comprising:
(a) providing dehydrated decidua tissue;
(b) providing an aqueous based pharmaceutically acceptable carrier;
(c) combining (a) and (b) and mixing until uniform; and
(d) dehydrating the resultant mixture, thereby forming the dehydrated composition;
wherein the aqueous based carrier has a water content of at least 50% w/w, wherein the dehydrated composition is a film or sheet, and wherein the film or sheet has a water content of less than 5% w/w or less than 3% w/w. In some embodiments, the dehydrating step (d) is conducted by evaporative air-drying or lyophilization.

In another aspect of the present invention, disclosed is a dehydrated composition comprising:
(a) dehydrated decidua tissue; and
(b) an aqueous based pharmaceutically acceptable carrier comprising:
   (i) one or more cellulose ethers, and
   (ii) one or more plasticizers,
wherein the aqueous based carrier has a water content of at least 50% w/w prior to dehydration of the composition, wherein the composition is dehydrated by evaporative air-drying or lyophilization, wherein the dehydrated composition is a film or sheet, and wherein the film or sheet has a water content of less than 5% w/w or less than 3% w/w. In some embodiments, the one or more cellulose ethers comprise hydroxyethyl cellulose (HEC) or hydroxypropyl cellulose (HPC), or mixtures thereof. In some embodiments, the one or more plasticizers comprise one or more polyethylene glycols. In some embodiments, the one or more polyethylene glycols comprise polyethylene glycol 600.

Also disclosed in the context of the present invention are the following embodiments 1 to 43. Embodiment 1 is a composition comprising dehydrated decidua tissue. Embodiment 2 is the composition of embodiment 1, wherein the dehydrated decidua tissue comprises decidua basalis tissue, decidua capsularis tissue, or decidua parietalis tissue, or any combination thereof. Embodiment 3 is the composition of any one of embodiments 1 or 2, wherein the dehydrated decidua tissue is non-viable. Embodiment 4 is the composition of any one of embodiments 1 to 3, wherein the dehydrated decidua tissue is a powder. Embodiment 5 is the composition of any one of embodiments 1 to 4, wherein the dehydrated decidua tissue is lyophilized. Embodiment 6 is the composition of any one of embodiments 1 to 5, wherein the water content of the dehydrated decidua tissue is less than 5% w/w, or less than 3% w/w. Embodiment 7 is the composition of any one of embodiments 1 to 6 further comprising a pharmaceutically acceptable carrier. Embodiment 8 is the composition of embodiment 7, wherein the pharmaceutically acceptable carrier is aqueous based, preferably wherein the composition comprises at least 50% w/w water. Embodiment 9 is the composition of any of any one of embodiments 1 to 8, wherein the pharmaceutically acceptable carrier comprises one or more cellulose ethers. Embodiment 10 is the composition of embodiment 9, wherein the cellulose ether is a non-ionic cellulose ether. Embodiment 11 is the composition of embodiment 10, wherein the non-ionic cellulose ether is selected from hydroxyethyl cellulose (HEC) or hydroxypropyl cellulose (HPC), or mixtures thereof. Embodiment 12 is the composition of any one of embodiments 1 to 11, wherein the pharmaceutical acceptable carrier further comprises one or more plasticizers. Embodiment 13 is the composition of embodiment 12, wherein the plasticizer is a hydrophilic polyol. Embodiment 14 is the composition of embodiment 13, wherein the hydrophilic polyol is a hydrophilic polymeric polyol. Embodiment 15 is the composition of embodiment 14, wherein the hydrophilic polymeric polyol is one or more polyethylene glycols. Embodiment 16 is the composition of embodiment 15, wherein the one or more polyethylene glycols comprises polyethylene glycol 600. Embodiment 17 is the composition of any one of embodiments 1 to 16, wherein the composition is dehydrated. Embodiment 18 is the composition of embodiment 17, wherein the composition is dehydrated by evaporative air-drying. Embodiment 19 is the composition of embodiment 18, wherein the evaporative air drying is room temperature evaporative air-drying under gravity convection conditions. Embodiment 20 is the composition of embodiment 17, wherein the composition is dehydrated by lyophilization. Embodiment 21 is the composition of any one of embodiments 17 to 20, wherein the water content of the dehydrated composition is less than 5% w/w, or less than 3% w/w. Embodiment 22 is the composition of any one of embodiments 17 to 21, wherein the dehydrated composition is a sheet or film. Embodiment 23 is the composition of embodiment 22, wherein the sheet or film has a thickness of from about 0.1 mm to about 25 mm, or from about 0.5 to about 10 mm, or from about 1 mm to about 5 mm. Embodiment 24 is the composition of any one of embodiments 17 to 23, wherein the concentration of decidua tissue is from about 1% to about 75% w/w, or from about 4% to about 50% w/w of the dehydrated composition. Embodiment 25 is the composition of any one of embodiments 17 to 24, wherein the dehydrated composition is capable of forming a hydrogel when the dehydrated composition is contacted with water or other aqueous medium. Embodiment 26 is the composition of any one of embodiments 17 to 25, wherein the dehydrated composition is opaque. Embodiment 27 is the composition of any one of embodiments 17 to 25, wherein the dehydrated composition is transparent.

Embodiment 28 is a method of treating a wound in a subject, the method comprising applying the composition of any one of embodiments 1 to 27 to the wound. Embodiment 29 is the method of embodiment 28, wherein the composition is applied in conjunction with applying another wound dressing. Embodiment 30 is the method of any one of embodiments 28 or 29, wherein the composition is a powder. Embodiment 31 is the method of any one of embodiments 28 to 30, wherein the wound is a dermal wound. Embodiment 32 is the method of embodiment 31, wherein the dermal wound is a partial thickness or a full thickness wound. Embodiment 33 is the method of any one of embodiments 31 or 32, wherein the dermal wound is a burn, a chronic wound, or an acute wound. Embodiment 34 is the method of embodiment 33, wherein the dermal wound is a burn and is selected from a superficial (first degree) burn, a partial thickness (second degree) burn, a full thickness (third degree) burn, or a radiation burn. Embodiment 35 is the method of embodiment 33, wherein the dermal wound is a chronic wound and is selected from a dermal ulcer, a diabetic ulcer, a diabetic foot ulcer, a venous ulcer, a venous leg ulcer, an arterial ulcer, an arterial leg ulcer, a decubitus ulcer, a stasis ulcer, an ischemic ulcer, a vascular ulcer, a pressure ulcer (stage I-IV), a podiatric wound, a draining wound, a tunneling wound, or an undermining wound. Embodiment 36 is the method of embodiment 33, wherein the dermal wound is an acute wound and is selected from a trauma wound, a laceration, an abrasion, a skin tear, a skin lesion, a blister, a surgical incision, a donor skin site, a skin graft, a laser surgery wound, a Mohs surgery wound, or a dehisced wound.

Embodiment 37 is a method of treating a wound in a subject, the method comprising applying a composition comprising decidua tissue to the wound.

Embodiment 38 is a method of making a dehydrated composition comprising dehydrated decidua tissue, the method comprising:
(a) providing dehydrated decidua tissue;
(b) providing an aqueous based pharmaceutically acceptable carrier;
(c) combining (a) and (b) and mixing until uniform; and
(d) dehydrating the resultant mixture, thereby forming the dehydrated composition; wherein the aqueous based carrier has a water content of at least 50% w/w, wherein the dehydrated composition is a film or sheet, and wherein the film or sheet has a water content of less than 5% w/w or less than 3% w/w. Embodiment 39 is the method of embodiment 38, wherein the dehydrating step (d) is conducted by evaporative air-drying or lyophilization.

Embodiment 40 is a dehydrated composition comprising:
(a) dehydrated decidua tissue; and
(b) an aqueous based pharmaceutically acceptable carrier comprising:
   (i) one or more cellulose ethers, and
   (ii) one or more plasticizers,
wherein the aqueous based carrier has a water content of at least 50% w/w prior to dehydration of the composition, wherein the composition is dehydrated by evaporative air-drying or lyophilization, wherein the dehydrated composition is a film or sheet, and wherein the film or sheet has a water content of less than 5% w/w or less than 3% w/w. Embodiment 41 is the composition of embodiment 40, wherein the one or more cellulose ethers comprise hydroxyethyl cellulose (HEC) or hydroxypropyl cellulose (HPC), or mixtures thereof. Embodiment 42 is the composition of any one of embodiments 40 or 41, wherein the one or more plasticizers comprise one or more polyethylene glycols. Embodiment 43 is the composition of embodiment 42, wherein the one or more polyethylene glycols comprise polyethylene glycol 600.

The term “subject” as used herein means a vertebrate animal and includes mammals which includes human beings. In some preferred aspects, the subject is a human.

The term “body” as used herein means the body of a subject.

The term “pieces” as used herein with respect to decidua tissue means tissue which has been subject to a disruption process, such as shearing, mincing, dicing, chopping, cutting, homogenizing, macerating, or crushing, in which small individual pieces of tissue are formed. Decidua pieces can further be dehydrated, dried, and/or lyophilized. The disruption process can occur before or after dehydration, evaporative air-drying, or lyophilization. The pieces can be uniform or irregular in size.

The term “powder” as used herein means dry, fine individual particles. The particles can be uniform or irregular in size. The particle size can be that described below in this specification.

The term “immunoprivileged” as used herein means a relatively high resistance against promoting an immune response.

The term “non-immunogenic” as used herein means not promoting an immune response.

The terms “room temperature” or “RT” as used herein mean a temperature of 20°-25° C.

The terms “optional” or “optionally” as used herein mean that the subsequently described event, circumstance, or material may or may not occur or be present, and that the description includes instances where the event, circumstance, or material occurs or is present and instances where it does not occur or is not present.

The terms “about” or “approximately” as used herein are defined as being close to as understood by one of skill in the art, and in one non-limiting embodiment the terms are defined to be within 10%, preferably within 5%, more preferably within 1%, and most preferably within 0.5% of an associated disclosed value. The terms may be removed from the associated disclosed value and the exact value may be used instead.

When the lower limit value of a given percentage range does not include the % symbol and/or the percentage type (e.g., w/w, v/v, etc.), then the percentage type for the lower limit value is the same as for the upper limit value of the given percentage range. For example, the percentage range of “0.01 to 0.5% w/w” means “0.01% w/w to 0.5% w/w.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) as used herein are inclusive or open-ended and do not exclude additional, unrecited elements or method steps.

The use of the word “a” or “an” when used in conjunction with the terms “comprising”, “having”, “including”, or “containing” (or any variations of these words) may mean “one,” but it is also consistent with the meaning of “one or more.” “at least one,” and “one or more than one.”

For purposes of this application, a number value with one or more decimal places can be rounded to the nearest whole number using standard rounding guidelines, i.e., round up if the number being rounded is 5, 6, 7, 8, or 9; and round down if the number being rounded is 0, 1, 2, 3, or 4. For example, 0.42 can be rounded to 0.4.

The compositions and methods for their use can “comprise.” “consist essentially of,” or “consist of” any of the ingredients or steps disclosed throughout the specification. With respect to the transitional phrases “consist essentially of” or “consisting essentially of,” in one non-limiting aspect, a basic and novel characteristic of the compositions and methods of the present invention are their abilities to treat wounds and/or repair tissue in a subject with composition or dehydrated compositions comprising decidua tissue, preferably with compositions comprising dehydrated decidua tissue.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a photomicrograph of lyophilized decidua tissue powder. 1 square=250 microns.

FIG. 2A is a photo of lot 0055-0114L01 dehydrated film composition containing lyophilized decidua tissue powder-air dried.

FIG. 2B is a photo of lot 0055-0127L01 dehydrated film composition containing lyophilized decidua tissue powder-air dried.

FIG. 2C is a photo of lot 0055-0225L01 dehydrated film composition containing lyophilized decidua tissue powder-evaporative air-dried.

FIG. 2D is a photo of lot 0055-0331L01 dehydrated film composition containing lyophilized decidua tissue powder-evaporative air-dried.

FIG. 3A is a photo of lot 0055-0114L01 dehydrated sheet composition containing lyophilized decidua tissue powder-lyophilized.

FIG. 3B is a photo of lot 0055-0127L01 dehydrated sheet composition containing lyophilized decidua tissue powder-lyophilized.

FIG. 3C is a photo of lot 0055-0225L01 dehydrated sheet composition containing lyophilized decidua tissue powder-lyophilized.

FIG. 3D is a photo of lot 0055-0331L01 dehydrated sheet composition containing lyophilized decidua tissue powder-lyophilized.

FIG. 4 is a photomicrograph of the cell culture plate of lyophilized decidua tissue powder after culturing with hMSCs.

FIG. 5 is a photomicrograph of the cell culture plate of lyophilized decidua tissue powder after differentiation of the hMSCs to adipocytes.

FIG. 6A is a photomicrograph of live/dead cell stain of cell culture of lot 0055-0114L01 dehydrated film composition containing lyophilized decidua tissue powder-air dried, at Day 2.

FIG. 6B is a photomicrograph of live/dead cell stain of cell culture of lot 0055-0114L01 dehydrated film composition containing lyophilized decidua tissue powder-air dried, at Day 4.

FIG. 6C is a photomicrograph of live/dead cell stain of cell culture of lot 0055-0114L01 dehydrated film composition containing lyophilized decidua tissue powder-air dried, at Day 8.

FIG. 6D is a photomicrograph of live/dead cell stain of cell culture of lot 0055-0114L01 dehydrated film composition containing lyophilized decidua tissue powder-air dried, at Day 16.

FIG. 7A is a photomicrograph of live/dead cell stain of cell culture of lot 0055-0114L01 dehydrated sheet composition containing lyophilized decidua tissue powder-lyophilized, at Day 2.

FIG. 7B is a photomicrograph of live/dead cell stain of cell culture of lot 0055-0114L01 dehydrated sheet composition containing lyophilized decidua tissue powder-lyophilized, at Day 4.

FIG. 7C is a photomicrograph of live/dead cell stain of cell culture of lot 0055-0114L01 dehydrated sheet composition containing lyophilized decidua tissue powder-lyophilized, at Day 8.

FIG. 7D is a photomicrograph of live/dead cell stain of cell culture of lot 0055-0114L01 dehydrated sheet composition containing lyophilized decidua tissue powder-lyophilized, at Day 16.

FIG. 8A is a photomicrograph of live/dead cell stain of cell culture of lot 0055-0127L01 dehydrated film composition containing lyophilized decidua tissue powder-air dried, at Day 2.

FIG. 8B is a photomicrograph of live/dead cell stain of cell culture of lot 0055-0127L01 dehydrated film composition containing lyophilized decidua tissue powder-air dried, at Day 4.

FIG. 8C is a photomicrograph of live/dead cell stain of cell culture of lot 0055-0127L01 dehydrated film composition containing lyophilized decidua tissue powder-air dried, at Day 8.

FIG. 8D is a photomicrograph of live/dead cell stain of cell culture of lot 0055-0127L01 dehydrated film composition containing lyophilized decidua tissue powder-air dried, at Day 16.

FIG. 9A is a photomicrograph of live/dead cell stain of cell culture of lot 0055-0127L01 dehydrated film composition containing lyophilized decidua tissue powder-lyophilized, at Day 2.

FIG. 9B is a photomicrograph of live/dead cell stain of cell culture of lot 0055-0127L01 dehydrated film composition containing lyophilized decidua tissue powder-lyophilized, at Day 4.

FIG. 9C is a photomicrograph of live/dead cell stain of cell culture of lot 0055-0127L01 dehydrated film composition containing lyophilized decidua tissue powder-lyophilized, at Day 8.

FIG. 9D is a photomicrograph of live/dead cell stain of cell culture of lot 0055-0127L01 dehydrated film composition containing lyophilized decidua tissue powder-lyophilized, at Day 16.

FIG. 10A is a photomicrograph of live/dead cell stain of cell culture of lot 0055-0225L01 dehydrated film composition containing lyophilized decidua tissue powder-air dried, at Day 6.

FIG. 10B is a photomicrograph of live/dead cell stain of cell culture of lot 0055-0225L01 dehydrated film composition containing lyophilized decidua tissue powder-air dried, at Day 13.

FIG. 10C is a photomicrograph of live/dead cell stain of cell culture of lot 0055-0225L01 dehydrated film composition containing lyophilized decidua tissue powder-air dried, at Day 19.

FIG. 10D is a photomicrograph of live/dead cell stain of cell culture of lot 0055-0225L01 dehydrated film composition containing lyophilized decidua tissue powder-air dried, at Day 27.

FIG. 11A is a photomicrograph of live/dead cell stain of cell culture of lot 0055-0225L01 dehydrated film composition containing lyophilized decidua tissue powder-lyophilized, at Day 6.

FIG. 11B is a photomicrograph of live/dead cell stain of cell culture of lot 0055-0225L01 dehydrated film composition containing lyophilized decidua tissue powder-lyophilized, at Day 13.

FIG. 11C is a photomicrograph of live/dead cell stain of cell culture of lot 0055-0225L01 dehydrated film composition containing lyophilized decidua tissue powder-lyophilized, at Day 13.

FIG. 11D is a photomicrograph of live/dead cell stain of cell culture of lot 0055-0225L01 dehydrated film composition containing lyophilized decidua tissue powder-lyophilized, at Day 27.

FIG. 12A is a photomicrograph of live/dead cell stain of cell culture of Oasis® Burn Matrix at Day 7.

FIG. 12B is a photomicrograph of live/dead cell stain of cell culture of DermaGinate Ag™ Calcium Alginate Dressing at Day 7.

FIG. 12C is a photomicrograph of live/dead cell stain of cell culture of Healadex® Dressing at Day 7.

FIG. 12D is a photomicrograph of live/dead cell stain of cell culture of Mepitel® One Dressing at Day 7.

FIG. 12E is a photomicrograph of live/dead cell stain of cell culture of Integra® Dermal Regeneration Template at Day 7.

### DETAILED DESCRIPTION OF THE INVENTION

The present invention relates to compositions and wound dressings comprising decidua tissue and uses of these compositions for the treatment of wounds including dermal wounds. The compositions disclosed herein are also useful for the repair of tissue such as soft tissue including, but not limited to tendons, ligaments, cartilage, and other connective tissue. The decidua tissue compositions can further comprise a carrier, such as a pharmaceutically acceptable carrier. Surprisingly, the compositions of decidua tissue disclosed herein have shown to provide an unexpectedly greater beneficial environment for cellular attachment and proliferation of mesenchymal cells in culture as compared to many commercially available wound dressing products. Thus, the decidua tissue compositions described in this disclosure provide superior conditions to support the wound healing process versus existing wound dressing products. The decidua has long been considered a disposable and discardable material with little or no value and therefore, can be obtained at little or no cost. Thus, using decidua tissue as a component of the compositions of the present invention provides for a cost-effective product which is economical to produce.

### I. Compositions

### A. Decidua Tissue

The decidua is a mucosal tissue of the uterus that forms in preparation for pregnancy. The decidua is comprised of three parts: decidua basalis, decidua capsularis, and decidua parietalis. The decidua basalis is the maternal part of the placenta. The decidua is generally discarded even if the remaining placental tissue is to be processed into placental products. The inventors of the present application have discovered that decidua tissue, which is generally discarded, can now be used in a purposeful wound dressing product with superior biological properties with respect to supporting the wound healing process by allowing cellular proliferation and migration, vascular ingrowth, and/or the formation of granulation tissues.

The decidua tissue of the compositions disclosed herein can comprise decidua basalis tissue, decidua capsularis tissue, or decidua parictalis tissue, or any combinations thereof. In some embodiments, the decidua tissue comprises decidua basalis tissue. In some embodiments, the decidua tissue comprises decidua basalis tissue. In some embodiments, the decidua tissue comprises decidua capsularis tissue. In some embodiments, the decidua tissue is decidua capsularis tissue. In some embodiments, the decidua tissue comprises decidua parietalis tissue. In some embodiments, the decidua tissue is decidua parietalis tissue. In some embodiments, the decidua tissue comprises the whole decidua, that is, all three parts of the decidua, i.e., basalis tissue, decidua capsularis tissue and decidua parietalis tissue. In some embodiments, the decidua tissue comprises decidua basalis tissue and decidua capsularis tissue but not decidua parietalis tissue. In some embodiments, the decidua tissue comprises decidua basalis tissue and decidua parietalis tissue but not decidua capsularis tissue. In some embodiments, the decidua tissue comprises decidua capsularis tissue and decidua parietalis tissue but not decidua basalis tissue. In some embodiments, the decidua tissue does not contain amniotic tissue, chorionic tissue, and/or umbilical cord tissue. The decidua tissue can be dehydrated. The dehydration of the decidua tissue can be conducted by any suitable means known to one of skill in the art, including, but not limited to lyophilizing (freeze-drying), evaporative air drying (e.g., thermal drying with heated air under gravity convection or forced air convection conditions; or drying under ambient or room temperature conditions under gravity convection or forced air convection conditions), indirect drying (e.g., vacuum drying), contact drying (e.g., drying through contact with a heated material or source), dielectric drying (e.g., use of radiofrequency or microwaves), infrared drying, and/or supercritical drying. The dehydrated decidua tissue is considered “dehydrated” or “dry” when it has a water content (moisture content) of less than 15% w/w, or less than 14% w/w, or less than 13% w/w, or less than 12% w/w, or less than 10% w/w, or less than 9% w/w, or less than 8% w/w, or less than 7% w/w, or less than 6% w/w, or less than 5% w/w, or less than 4% w/w, or less than 3% w/w, or less than 2% w/w, less than 1% w/w, or less than 0.5% w/w. In some embodiments the water content is less than 5% w/w, or less than 4% w/w, or less than 3% w/w. The dehydrated decidua tissue can, for example, have a water content of 0, 1, 2, 3, 4, 5, 6, 7, 8, 9, 10, 11, 12, 13, or 14% w/w, or any percentage derivable therein. The water content (moisture content) of the dehydrated tissue can be determined by methods known to one of skill in the art such as by Karl Fischer titration or by oven drying. In a preferred embodiment, the dehydrated decidua tissue is lyophilized decidua tissue. The decidua tissue can be non-viable, meaning that no viable cells are present in or on the tissue. The decidua tissue can be rendered non-viable by any suitable means known to one of skill in the art, including, but not limited to cell lysis, sterilization, irradiation, or cell removal by enzymatic or physical means. In some embodiments, the decidua tissue is non-viable. In some embodiments the compositions of the present invention and/or the decidua tissue of the present invention are sterilized. In some embodiments, the decidua tissue is viable meaning that the decidua tissue comprises viable cells. The viable cells can be native cells or exogenous cells, or mixtures thereof. The decidua tissue or dehydrated decidua tissue, e.g., evaporative air-dried or lyophilized decidua tissue, can be in the form of pieces or powder. In some embodiments, the decidua tissue is dehydrated by lyophilization resulting in lyophilized decidua tissue. In some embodiments, the decidua tissue is rendered non-viable by cell lysis. In some embodiments, the decidua tissue is in powder form. In some embodiments, the decidua tissue comprises decidua basalis tissue and is dehydrated, non-viable, and in powder form. In some embodiments, the decidua tissue comprises decidua basalis tissue and is lyophilized, non-viable, and in powder form. In some embodiments, the decidua tissue or dehydrated decidua tissue powder is non-immunogenic and/or immunoprivileged.

The dehydrated, e.g., evaporative air-dried or lyophilized, decidua tissue powder can have a particle size of 90% less than 250 microns. The dehydrated decidua tissue powder can have a particle size or particle size distribution (mean particle size) of from about 425 to about 1000 microns, or from about 216 to about 425 microns, or less than about 216 microns. The dehydrated decidua tissue powder can have a particle size of from about 1 to about 1000 microns, or from about 1 to about 500 microns, or from about 1 to about 250 microns, or from about 50 to about 1000 microns, or from about 50 to about 500 microns, or from about 50 to about 250 microns, or from about 100 to about 1000 microns, or from about 100 to about 500 microns, or from about 100 to about 250 microns, or from about 200 to about 1000 microns, or from about 200 to about 500 microns, or from about 200 to about 250 microns, or from about 400 to about 1000 microns, or from about 400 to about 500 microns. The particle size can be determined by particle sizing methods known to one of skill in the art such as, but not limited to microscopic analysis, stacked sieving methods, and particle size analyzers. Particle size analyzers can employ light obscuration methods or laser diffraction methods. Suitable particle size analyzers include but are not limited to analyzers made by Malvern such as the Malvern Mastersizer™, AccuSizer, Shimadzu Corporation, and Beckman Coulter. A particle size distribution can be expressed as a mean particle size based on number distribution or volume distribution.

The concentration of decidua tissue, dehydrated decidua tissue, lyophilized decidua tissue, or evaporative air-dried decidua tissue in the composition can be at an amount effective to provide an environment to supporting the wound healing process by allowing cellular migration, vascular ingrowth, and the formation of granulation tissues when the composition is applied to a wound or damaged tissue as demonstrated in vitro by the cellular attachment and proliferation of mesenchymal cells in culture with the composition.

The concentrations of decidua tissue, dehydrated decidua tissue, lyophilized decidua tissue, or evaporative air-dried decidua tissue in the compositions in the hydrous state disclosed herein can be from about 0.05% to about 50% w/w, or from about 0.05% to about 40% w/w, or from about 0.05% to about 30% w/w, or from about 0.05% to about 25% w/w, or from about 0.05% to about 20% w/w, or from about 0.05% to about 15% w/w, or from about 0.05% to about 10% w/w, or from about 0.05% to about 5% w/w, or from about 0.05% to about 4% w/w, or from about 0.05% to about 3% w/w, or from about 0.05% to about 2% w/w, or from about 0.05% to about 1% w/w, or from about 0.1% to about 50% w/w, or from about 0.1% to about 40% w/w, or from about 0.1% to about 30% w/w, or from about 0.1% to about 25% w/w, or from about 0.1% to about 20% w/w, or from about 0.1% to about 15% w/w, or from about 0.1% to about 10% w/w, or from about 0.1% to about 5% w/w, or from about 0.1% to about 4% w/w, or from about 0.1% to about 3% w/w, or from about 0.1% to about 2% w/w, or from about 0.1% to about 1% w/w, or from about 0.5% to about 50% w/w, or from about 0.5% to about 40% w/w, or from about 0.5% to about 30% w/w, or from about 0.5% to about 25% w/w, or from about 0.5% to about 20% w/w, or from about 0.5% to about 15% w/w, or from about 0.5% to about 10% w/w, or from about 0.5% to about 5% w/w, or from about 0.5% to about 4% w/w, or from about 0.5% to about 3% w/w, or from about 0.5% to about 2% w/w, or from about 0.5% to about 1% w/w, or from about 1% to about 50% w/w, or from about 1% to about 40% w/w, or from about 1% to about 30% w/w, or from about 1% to about 25% w/w, or from about 1% to about 20% w/w, or from about 1% to about 15% w/w, or from about 1% to about 10% w/w, or from about 1% to about 5% w/w, or from about 1% to about 4% w/w, or from about 1% to about 3% w/w, or from about 1% to about 2% w/w, of the total composition for compositions in the hydrous state.

The concentrations of decidua tissue, dehydrated decidua tissue, lyophilized decidua tissue, or evaporative air-dried decidua tissue in the compositions in the dehydrated state disclosed herein can be from about 1% to about 75% w/w, or from about 1% to about 60% w/w, or from about 1% to about 50% w/w, or from about 2% to about 50% w/w, or from about 3% to about 50% w/w, or from about 4% to about 50% w/w, or from about 5% to about 50% w/w, or from about 6% to about 50% w/w, or from about 7% to about 50% w/w, or from about 8% to about 50% w/w, or from about 9% to about 50% w/w, or from about 10% to about 50% w/w, or from about 15% to about 50% w/w, or from about 20% to about 50% w/w, or from about 1% to about 40% w/w, or from about 2% to about 40% w/w, or from about 3% to about 40% w/w, or from about 4% to about 40% w/w, or from about 5% to about 40% w/w, or from about 6% to about 40% w/w, or from about 7% to about 40% w/w, or from about 8% to about 40% w/w, or from about 9% to about 40% w/w, or from about 10% to about 40% w/w, or from about 15% to about 40% w/w, or from about 20% to about 40% w/w, or from about 1% to about 30% w/w, or from about 2% to about 30% w/w, or from about 3% to about 30% w/w, or from about 4% to about 30% w/w, or from about 5% to about 30% w/w, or from about 6% to about 30% w/w, or from about 7% to about 30% w/w, or from about 8% to about 30% w/w, or from about 9% to about 30% w/w, or from about 10% to about 30% w/w, or from about 15% to about 30% w/w, or from about 20% to about 30% w/w, of the total composition for compositions in the dehydrated state.

The decidua tissue can be processed as described in the following non-limiting exemplified embodiment. The unprocessed decidua tissue is added to water or buffer solution, such as phosphate buffered saline (PBS), then added to a suitable homogenizer, such as a blender or other type of homogenizer, and homogenized to form pieces. The homogenized tissue pieces are collected by centrifugation techniques. A suitable lysing buffer, such as ACK RBC Lysing Buffer, is added to the tissue and allowed to contact the tissue for a time sufficient to lyse the cells. The lysed treated tissue is then washed with water or a buffer solution and collected by centrifugation techniques. The tissue is then lyophilized (freeze-dried) using a lyophilizer or other suitable freeze-drying technique. The lyophilized tissue is then processed into a powder form using a suitable mill, grinder, or dissociator. In some embodiments, the decidua tissue or dehydrated decidua tissue powder is non-immunogenic and/or immunoprivileged. In some embodiments, the compositions or dehydrated compositions disclosed herein comprising decidua tissue or dehydrated decidua tissue are non-immunogenic and/or immunoprivileged.

### B. Carriers

The compositions comprising decidua tissue disclosed herein can further comprise a carrier, such as a pharmaceutically acceptable carrier. Non-limiting examples of carriers include lotions, creams, emulsions, ointments, gels, hydrogels, pastes, solutions, aerosol sprays, aerosol foams, non-aerosol sprays, non-aerosol foams, powders, liquid solutions, liquid suspensions, films, and sheets. The carrier or pharmaceutically acceptable carrier can be aqueous based, anhydrous, hydrophilic, hydrophobic, or anhydrous hydrophilic. For example, an aqueous based carrier or pharmaceutically acceptable carrier can be an aqueous based gel or hydrogel. The compositions can be prepared by incorporating the decidua tissue into the carrier using methods known to those of skill in the art. Such methods can include the use of various types of mixers, blenders, and homogenizers. The compositions of decidua tissue and a carrier may be impregnated in gauzes, bandages, or other wound dressing materials. In some embodiments, the decidua tissue, dehydrated decidua tissue, lyophilized decidua tissue, or evaporative air-dried decidua tissue is in the form of pieces or powder and is distributed uniformly within the carrier. In some embodiments, the carrier is a pharmaceutically acceptable carrier. In some embodiments, the carrier or the pharmaceutically acceptable carrier is aqueous based. The compositions can be aqueous based, anhydrous, or dehydrated. Non-limiting examples of dehydrated compositions comprising decidua tissue and a carrier include films, sheets, or powders. In some aspects of the present invention, an aqueous-based composition, carrier, gel, or hydrogel can include at least 30%, 40%, 50%, 60%, 70%, 80%, or 90% w/w or more of water, preferably at least 50% w/w water.

i. Dehydrated Film and Sheet Compositions and Methods of Preparation

In another aspect of the present invention, dehydrated (dry) compositions in the form of dehydrated films or sheets are disclosed. The films or sheets can comprise dehydrated decidua tissue, evaporative air-dried decidua tissue, or lyophilized decidua tissue in pieces or in powder form, and a carrier. The terms “films” and “sheets” as used herein can be used interchangeably, but generally a film is thinner than a sheet. The carrier or pharmaceutically acceptable carrier of the compositions can be a dehydrated film or sheet. Exemplary methods for preparing compositions of dehydrated films or sheets comprise dehydrating compositions of aqueous gels or hydrogels comprising decidua tissue or dehydrated decidua tissue. The aqueous gel or hydrogel comprising decidua tissue or dehydrated decidua tissue can be poured into a suitable mold such as a petri dish prior to dehydration. The aqueous-based gel or hydrogel comprising the decidua tissue or dehydrated decidua tissue can be dehydrated by any suitable means including but not limited to lyophilizing (freeze-drying), evaporative air drying (e.g., thermal drying with heated air under gravity convection or forced air convection conditions; or drying under ambient or room temperature conditions under gravity convection or forced air convection conditions), indirect drying (e.g., vacuum drying), contact drying (e.g., drying through contact with a heated material or source), dielectric drying (e.g., use of radiofrequency or microwaves), infrared drying, and/or supercritical drying. The resultant film or sheet from evaporative air-drying can be referred to as a xerogel. Evaporative air drying can be accomplished under gravity convection or forced air convention conditions at ambient or room temperature or higher temperatures. Drying using heated air can be accomplished in a heating chamber at temperatures above room temperature with gravity convection or with forced air convection conditions. Drying under gravity convection at ambient or room temperature can be accomplished in a chamber or in an open air space such as on a table or bench. In some embodiments, the evaporative air-drying is thermal evaporative air-drying with heated air under gravity convection or forced air convection conditions. In other embodiments, the evaporative air-drying is ambient or room temperature evaporative air-drying under gravity convection or forced air convection conditions. In still other embodiments, the evaporative air-drying is room temperature evaporative air-drying under gravity convection conditions. Lyophilizing, aka freeze-drying, can be accomplished by first freezing the aqueous gel or hydrogel in a freezer, e.g., at −80° C., then drying in a lyophilizer; or by freezing and drying in a lyophilizer. In some embodiments the gel or hydrogel comprising decidua tissue or dehydrated decidua tissue is dehydrated by evaporative air-drying or lyophilization. The film or sheet is considered “dehydrated” or “dry” when it has a water content (moisture content) of less than 15% w/w, or less than 14% w/w, or less than 13% w/w, or less than 12% w/w, or less than 10% w/w, or less than 9% w/w, or less than 8% w/w, or less than 7% w/w, or less than 6% w/w, or less than 5% w/w, or less than 4% w/w, or less than 3% w/w, or less than 2% w/w, less than 1% w/w, or less than 0.5% w/w. In some embodiments the water content is less than 5% w/w, or less than 4% w/w, or less than 3% w/w. The dehydrated film or sheet can, for example, have a water content of 0%, 1%, 2%, 3%, 4%, 5%, 6%, 7%, 8%, 9%, 10%, 11%, 12%, 13%, or 14% w/w, or any percentage derivable therein. The water content (moisture content) of the dehydrated film or sheet can be determined by methods known to one of skill in the art such as by Karl Fischer titration or by oven drying. For example, the dehydrated film or sheet can be placed in a 65° C. oven for 3 minutes at atmospheric pressure and measuring the weight loss after incubation to determine moisture content.

The decidua tissue pieces or powder, dehydrated decidua tissue pieces or powder, evaporative air-dried decidua tissue pieces or powder, or lyophilized decidua tissue pieces or powder is uniformly dispersed within the aqueous gel or hydrogel carrier prior to the dehydration step to form the dehydrated film or sheet. A rheology modifier, such as a gellant, can be used to form the aqueous gel or hydrogel prior to dehydration by the addition of the rheology modifier to water or an aqueous medium. The decidua tissue pieces or powder can be added prior to or after the addition of the rheology modifier. The decidua tissue pieces or powder can first be suspended in water or a buffer solution, such as PBS, prior to its addition to wet the tissue to form a slurry then the slurry can further be homogenized with a homogenizer such as a Tissue Tearor™ to facilitate incorporation of the tissue into the aqueous gel or hydrogel carrier. Mixing of the decidua tissue pieces or powder and rheology modifier in the water or aqueous medium can be conducted using methods known to one of skill in the art using suitable mixing equipment known to one of skill in the art, such as propeller mixers, dissolvers, homogenizers, and the like. Suitable rheology modifiers include, but are not limited to cellulose ethers, microcrystalline cellulose, acrylic polymers, alginates, gums, and organoclays, examples of which can be found in “Rheology Modifiers Handbook, Practical Use and Application,” William Andrew Publishing, 2000, herein incorporated by reference. In some embodiments, the carrier comprises one or more rheology modifiers. In some embodiments, the rheology modifier is a cellulose ether. In some embodiments, the carrier comprises one or more cellulose ethers.

Cellulose ethers include non-ionic and anionic cellulose ethers and are available in a variety of viscosity grades. Nonionic cellulose ethers are high-molecular-weight compounds that can be made by replacing the hydrogen atoms of hydroxyl groups in the glucose units of cellulose with alkyl or hydroxylalkyl groups. Non-limiting examples of non-ionic alkyl cellulose ethers include methyl cellulose (MC), ethyl cellulose (EC), and ethyl methyl cellulose (EMC). Non-limiting examples of non-ionic hydroxyalkyl cellulose ethers include hydroxyethyl cellulose (HEC), hydroxypropyl cellulose (HPC), hydroxymethyl cellulose (HMC), hydroxypropylmethyl cellulose (HPMC), ethylhydroxyethyl cellulose (EHEC), hydroxyethylmethy cellulose (HEMC), methylhydroxyethyl cellulose (MHEC), methylhydroxypropylcellulose (MHPC), and hydroxyethylcarboxymethyl cellulose (HECMC). There are a wide range of commercial sources for each of these cellulose ethers (e.g., Dow Chemical Company, Ashland, Spectrum Chemical). HEC is available under the name NATROSOL™ from Ashland. HPC is available under the name KLUCEL™ from Ashland. HPMC (also known as hypromellose) is available under the name BENECEL™ from Ashland. An example of an anionic cellulose ether is sodium carboxymethyl cellulose (CMC) available commercially under the name AQUALON™ from Ashland. The cellulose ethers are available in cosmetic grades and pharmaceutical grades (USP/NF) and are suitable for use in the disclosed compositions. The concentration of the cellulose ether in the composition can vary as needed to achieve a particular characteristic of the dehydrated film or sheet. The concentration of the cellulose ether in the composition (cumulative concentration if more than one cellulose ether is present) can be from about 20% to about 80% w/w, or from about 30% to about 80% w/w, or from about 40% to about 80% w/w for a composition in the dehydrated state. The carrier can comprise one or more cellulose ethers. In some embodiments, the carrier comprises one or more cellulose ethers. In some embodiments, the cellulose ether is a non-ionic cellulose ether. In some embodiments, the non-ionic cellulose ether is hydroxyethyl cellulose (HEC) or hydroxypropyl cellulose (HPC), or mixtures thereof.

The carrier can further comprise one or more plasticizers, which can serve to enhance the desired physical properties, such as brittleness and flexibility, of dehydrated films or sheets. Non-limiting examples of plasticizers include hydrophilic polyols, hydrophilic polymeric polyols, propylene glycol, glycerol, polyethylene glycol (PEG), polypropylene glycol, poloxamers, and povidone. The concentration of the plasticizer in the composition can be from about 0% to about 30% w/w, or from about 5% to about 30% w/w, or from about 10% to about 25% w/w for the composition in the dehydrated state. In some embodiments, the plasticizer is a hydrophilic polyol. Suitable hydrophilic polyols are water-soluble, polar aliphatic alcohols with at least two hydroxyl groups, and can include polymeric polyols, e.g., polyethylene glycols and poloxamers. In some embodiments, the hydrophilic polyol is a hydrophilic polymeric polyol. In some embodiments, the hydrophilic polymeric polyol is one or more polyethylene glycols. In some embodiments, the polyethylene glycol is polyethylene glycol 600.

Polyethylene glycols (PEG) are homo-polymers of ethylene glycol and water represented by the formula:

H(OCH₂CH₂)ₙOH

in which n represents the average number of oxyethylene groups. Polyethylene glycols can be either liquid or solid at 25° C. depending on their molecular weights. The following non-limiting examples are suitable for use with the disclosed compositions and are described using U.S. Pharmacopeial Convention (USP) nomenclature: polyethylene glycol 200, polyethylene glycol 300, polyethylene glycol 400, polyethylene glycol 500, and polyethylene glycol 600. The following non-limiting examples of solid polyethylene glycols are suitable for use with the disclosed compositions and are described using USP nomenclature: polyethylene glycol 700, polyethylene glycol 800, polyethylene glycol 900, polyethylene glycol 1000, polyethylene glycol 1100, polyethylene glycol 1200, polyethylene glycol 1300, polyethylene glycol 1400, polyethylene glycol 1450, polyethylene glycol 1500, polyethylene glycol 1600, polyethylene glycol 1700, polyethylene glycol 1800, polyethylene glycol 1900, polyethylene glycol 2000, polyethylene glycol 2100, polyethylene glycol 2200, polyethylene glycol 2300, polyethylene glycol 2400, polyethylene glycol 2500, polyethylene glycol 2600, polyethylene glycol 2700, polyethylene glycol 2800, polyethylene glycol 2900, polyethylene glycol 3000, polyethylene glycol 3250, polyethylene glycol 3350, polyethylene glycol 3750, polyethylene glycol 4000, polyethylene glycol 4250, polyethylene glycol 4500, polyethylene glycol 4750, polyethylene glycol 5000, polyethylene glycol 5500, polyethylene glycol 6000, polyethylene glycol 6500, polyethylene glycol 7000, polyethylene glycol 7500, and polyethylene glycol 8000. Such liquid and solid polyethylene glycols are available commercially from the DOW Chemical Company under the CARBOWAX™ and SENTRY™ names and from the BASF Corporation under the PLURACARE® and PLURIOL® names. Polyethylene glycols are available in cosmetic grades and pharmaceutical grades (USP/NF) and are suitable for use in the disclosed compositions. The concentration of the polyethylene glycol in the composition (cumulative concentration if more than one polyethylene glycol is present) can be from about 0% to about 30% w/w, or from about 5% to about 30% w/w, or from about 10% to about 25% w/w for the composition in the dehydrated state. In some embodiments, the carrier comprises polyethylene glycol (PEG) 600. PEG 600 is available under the name PLURACARE® E 600 from BASF and under the name CARBOWAX™ SENTRY™ Polyethylene Glycol 600 NF from Dow.

The concentration of decidua tissue as pieces or powder, dehydrated decidua tissue as pieces or powder, evaporative air-dried decidua tissue pieces or powder, or lyophilized decidua tissue pieces or powder in the dehydrated film or sheet composition can be an amount effective to provide an environment to supporting the wound healing process by allowing cellular migration, vascular ingrowth, and the formation of granulation tissues when the composition is applied to a wound or damaged tissue as demonstrated in vitro by the cellular attachment and proliferation of mesenchymal cells in culture with the composition and a suitable culture medium such as MSC culture medium, e.g. DMEM+10% FBS. The concentration of decidua tissue pieces or powder, dehydrated decidua tissue pieces or powder, evaporative air-dried decidua tissue pieces or powder, or lyophilized decidua tissue pieces or powder in the dehydrated film or sheet composition can be from about 1% to about 75% w/w, or from about 1% to about 60% w/w, or from about 1% to about 50% w/w, or from about 2% to about 50% w/w, or from about 3% to about 50% w/w, or from about 4% to about 50% w/w, or from about 5% to about 50% w/w, or from about 6% to about 50% w/w, or from about 7% to about 50% w/w, or from about 8% to about 50% w/w, or from about 9% to about 50% w/w, or from about 10% to about 50% w/w, or from about 1% to about 40% w/w, or from about 2% to about 40% w/w, or from about 3% to about 40% w/w, or from about 4% to about 40% w/w, or from about 5% to about 40% w/w, or from about 6% to about 40% w/w, or from about 7% to about 40% w/w, or from about 8% to about 40% w/w, or from about 9% to about 40% w/w, or from about 10% to about 40% w/w, of the total composition for compositions in the dehydrated state. For example, the concentration of the decidua pieces or powder, dehydrated decidua tissue pieces or powder, evaporative air-dried decidua tissue pieces or powder, or lyophilized decidua tissue pieces or powder in the dehydrated film or sheet composition can be about 1%, or about 2%, or about 3%, about 4% or about 5%, or about 10%, or about 11%, or about 15%, or about 20%, or about 25%, or about 30%, or about 39%, or about 40%, or about 42%, or about 45%, or about 50%, or about 55%, or about 60% or about 65%, or about 70%, or about 75% w/w. In some embodiments, the dehydrated decidua tissue, evaporative air-dried decidua tissue, or lyophilized decidua tissue is in powder form.

Disclosed is a non-limiting exemplary method of a method of making a dehydrated composition comprising dehydrated decidua tissue, the method comprising:
(a) providing dehydrated decidua tissue;
(b) providing an aqueous based pharmaceutically acceptable carrier;
(c) combining (a) and (b) and mixing until uniform; and
(d) dehydrating the resultant mixture, thereby forming the dehydrated composition;
wherein the aqueous based carrier has a water content of at least 50% w/w, wherein the dehydrated composition is a film or sheet, and wherein the film or sheet has a water content of less than 5% w/w or less than 3% w/w. In some embodiments, the dehydrating step (d) is conducted by evaporative air-drying or lyophilization.

Disclosed is a non-limiting exemplary dehydrated composition comprising
(a) dehydrated decidua tissue; and
(b) an aqueous based pharmaceutically acceptable carrier comprising:
   (i) one or more cellulose ethers, and
   (ii) one or more plasticizers,
wherein the aqueous based carrier has a water content of at least 50% w/w prior to dehydration of the composition, wherein the composition is dehydrated by evaporative air-drying or lyophilization, wherein the dehydrated composition is a film or sheet, and wherein the film or sheet has a water content of less than 5% w/w or less than 3% w/w. In some embodiments, the one or more cellulose ethers comprise hydroxyethyl cellulose (HEC) or hydroxypropyl cellulose (HPC), or mixtures thereof. In some embodiments, the one or more plasticizers comprise one or more polyethylene glycols. In some embodiments, the one or more polyethylene glycols comprise polyethylene glycol 600.

A feature of the dehydrated film and sheet compositions disclosed herein is that they have the capability of forming a hydrogel when they come in contact with water or other aqueous medium, such as a buffer or saline solution, or moisture from the wound itself. Thus, when the dehydrated film or sheet composition is applied to a wound, the moisture from the wound or external source reconstitutes the composition back into a hydrogel. The resultant hydrogel can have a sufficient viscosity that it remains on the wound and does not drain off. The dehydrated film or sheet can be capable of not disintegrating as is the case with other film wound dressings, but rather forms a hydrogel that remains on the wound creating an environment and a physical matrix or scaffold that provides conditions to support the wound healing process by allowing cellular migration, vascular ingrowth, and the formation of granulation tissues. The amount of the cellulose ether and/or plasticizer within the dehydrated film or sheet composition can vary as needed to achieve a desired viscosity in the resultant hydrogel that is formed when the dehydrated film or sheet comes in contact with water or other aqueous medium. In some embodiments, the resultant hydrogel has a sufficient viscosity so that it will remain in a wound bed and not drain off the wound. The viscosity of the resultant hydrogel can be measured using a viscometer, such as a Brookfield viscometer. A preferred method of measuring the viscosity of the resultant hydrogel is using a Brookfield viscometer Model RV-DV2T cone & plate viscometer using cone spindle CP-52 at 0.5 RPM at RT and running for 1 minute. The sample for the viscosity measurement is prepared by contacting about 1 part by weight of the dehydrated film or sheet with about 20 parts of water or other aqueous medium such as normal saline solution. Viscosity values are reported in centipoise (cps). Viscosity values using the preferred method can be from about 100 cps to about 100,000 cps, or from about 100 cps to about 75,000 cps, or from about 100 cps to about 50,000 cps, or from about 100 cps to about 40,000 cps, of from about 100 cps to about 30,000 cps, or from about 100 cps to about 25,000 cps, or from about 100 cps to about 20,000 cps, or from about 100 cps to about 19,000 cps, or from about 100 cps to about 18,000 cps, or from about 100 cps to about 17,000 cps, or from about 100 cps to about 16,000 cps, or from about 100 cps to about 15,000 cps, or from about 100 cps to about 14,000 cps, or from about 100 cps, to about 13,000 cps, or from about 100 cps to about 12,000 cps, or from about 500 cps to about 100,000 cps, or from about 500 cps to about 75,000 cps, or from about 500 cps to about 50,000 cps, or from about 500 cps to about 40,000 cps, of from about 500 cps to about 30,000 cps, or from about 500 cps to about 25,000 cps, or from about 500 cps to about 20,000 cps, or from about 500 cps to about 19,000 cps, or from about 500 cps to about 18,000 cps, or from about 500 cps to about 17,000 cps, or from about 500 cps to about 16,000 cps, or from about 500 cps to about 15,000 cps, or from about 500 cps to about 14,000 cps, or from about 500 cps, to about 13,000 cps, or from about 500 cps to about 12,000 cps, or from about 1000 cps to about 100,000 cps, or from about 1000 cps to about 75,000 cps, or from about 1000 cps to about 50,000 cps, or from about 1000 cps to about 40,000 cps, of from about 1000 cps to about 30,000 cps, or from about 1000 cps to about 25,000 cps, or from about 1000 cps to about 20,000 cps, or from about 1000 cps to about 19,000 cps, or from about 1000 cps to about 18,000 cps, or from about 1000 cps to about 17,000 cps, or from about 1000 cps to about 16,000 cps, or from about 1000 cps to about 15,000 cps, or from about 1000 cps to about 14,000 cps, or from about 1000 cps, to about 13,000 cps, or from about 1000 cps to about 12,000 cps, or from about 5000 cps to about 100,000 cps, or from about 5000 cps to about 75,000 cps, or from about 5000 cps to about 50,000 cps, or from about 5000 cps to about 40,000 cps, of from about 5000 cps to about 30,000 cps, or from about 5000 cps to about 25,000 cps, or from about 5000 cps to about 20,000 cps, or from about 5000 cps to about 19,000 cps, or from about 5000 cps to about 18,000 cps, or from about 5000 cps to about 17,000 cps, or from about 5000 cps to about 16,000 cps, or from about 5000 cps to about 15,000 cps, or from about 5000 cps to about 14,000 cps, or from about 5000 cps, to about 13,000 cps, or from about 5000 cps to about 12,000 cps, or from about 10,000 cps to about 100,000 cps, or from about 10,000 cps to about 75,000 cps, or from about 10,000 cps to about 50,000 cps, or from about 10,000 cps to about 40,000 cps, of from about 10,000 cps to about 30,000 cps, or from about 10,000 cps to about 25,000 cps, or from about 10,000 cps to about 20,000 cps, or from about 10,000 cps to about 19,000 cps, or from about 10,000 cps to about 18,000 cps, or from about 10,000 cps to about 17,000 cps, or from about 10,000 cps to about 16,000 cps, or from about 10,000 cps to about 15,000 cps, or from about 10,000 cps to about 14,000 cps, or from about 10,000 cps, to about 13,000 cps, or from about 10,000 cps to about 12,000 cps, or from about 12,000 cps to about 100,000 cps, or from about 12,000 cps to about 75,000 cps, or from about 12,000 cps to about 50,000 cps, or from about 12,000 cps to about 40,000 cps, of from about 12,000 cps to about 30,000 cps, or from about 12,000 cps to about 25,000 cps, or from about 12,000 cps to about 20,000 cps, or from about 12,000 cps to about 19,000 cps, or from about 12,000 cps to about 18,000 cps, or from about 12,000 cps to about 17,000 cps, or from about 12,000 cps to about 16,000 cps, or from about 12,000 cps to about 15,000 cps, or from about 12,000 cps to about 14,000 cps, or from about 12,000 cps, to about 13,000 cps or from about 15,000 cps to about 100,000 cps, or from about 15,000 cps to about 75,000 cps, or from about 15,000 cps to about 50,000 cps, or from about 15,000 cps to about 40,000 cps, of from about 15,000 cps to about 30,000 cps, or from about 15,000 cps to about 25,000 cps, or from about 15,000 cps to about 20,000 cps, or from about 20,000 cps to about 100,000 cps, or from about 20,000 cps to about 75,000 cps, or from about 20,000 cps to about 50,000 cps, or from about 20,000 cps to about 40,000 cps, of from about 20,000 cps to about 30,000 cps, or from about 20,000 cps to about 25,000 cps.

The terms “films” and “sheets” as used herein can be used interchangeably, but generally a film is thinner than a sheet. The thickness of the films or sheets is a function of the amount of the hydrogel placed into a mold prior to dehydration, i.e., the more hydrogel in a given mold, will result in a thicker film or sheet after dehydrating. The thickness of the dehydrated film or sheet can be from about 0.1 mm to about 25 mm, or from about 0.1 to about 20 mm, or from about 0.1 to about 15 mm, or from about 0.1 to about 10 mm, or from about 0.1 to about 9 mm, or from about 0.1 to about 8 mm, or from about 0.1 to about 7 mm, or from about 0.1 to about 6 mm, or from about 0.1 to about 5 mm, or from about 0.1 to about 4 mm, or from about 0.1 to about 3 mm, or from about 0.1 to about 2 mm, or from about 0.1 to about 1.5 mm, or from about 0.1 to about 1 mm, or from about 0.2 mm to about 25 mm, or from about 0.2 to about 20 mm, or from about 0.2 to about 15 mm, or from about 0.2 to about 10 mm, or from about 0.2 to about 9 mm, or from about 0.2 to about 8 mm, or from about 0.2 to about 7 mm, or from about 0.2 to about 6 mm, or from about 0.2 to about 5 mm, or from about 0.2 to about 4 mm, or from about 0.2 to about 3 mm, or from about 0.2 to about 2 mm, or from about 0.2 to about 1.5 mm, or from about 0.2 to about 1 mm, or from about 0.3 mm to about 25 mm, or from about 0.3 to about 20 mm, or from about 0.3 to about 15 mm, or from about 0.3 to about 10 mm, or from about 0.3 to about 9 mm, or from about 0.3 to about 8 mm, or from about 0.3 to about 7 mm, or from about 0.3 to about 6 mm, or from about 0.3 to about 5 mm, or from about 0.3 to about 4 mm, or from about 0.3 to about 3 mm, or from about 0.3 to about 2 mm, or from about 0.3 to about 1.5 mm, or from about 0.3 to about 1 mm, or from about 0.4 mm to about 25 mm, or from about 0.4 to about 20 mm, or from about 0.4 to about 15 mm, or from about 0.4 to about 10 mm, or from about 0.4 to about 9 mm, or from about 0.4 to about 8 mm, or from about 0.4 to about 7 mm, or from about 0.4 to about 6 mm, or from about 0.4 to about 5 mm, or from about 0.4 to about 4 mm, or from about 0.4 to about 3 mm, or from about 0.4 to about 2 mm, or from about 0.4 to about 1.5 mm, or from about 0.4 to about 1 mm, or from about 0.5 mm to about 25 mm, or from about 0.5 to about 20 mm, or from about 0.5 to about 15 mm, or from about 0.5 to about 10 mm, or from about 0.5 to about 9 mm, or from about 0.5 to about 8 mm, or from about 0.5 to about 7 mm, or from about 0.5 to about 6 mm, or from about 0.5 to about 5 mm, or from about 0.5 to about 4 mm, or from about 0.5 to about 3 mm, or from about 0.5 to about 2 mm, or from about 0.5 to about 1.5 mm, or from about 0.5 to about 1 mm, or from about 1 to about 25 mm, or from about 1 to about 20 mm, or from about 1 to about 15 mm, or from about 1 to about 10 mm, or from about 1 to about 9 mm, or from about 1 to about 8 mm, or from about 1 to about 7 mm, or from about 1 to about 6 mm, or from about 1 to about 5 mm, or from about 1 to about 4 mm, or from about 1 to about 3 mm, or from about 1 to about 2 mm, or from about 1 to about 1.5 mm. The film or sheet can be cut or pre-cut in any size suitable for application to a wound or tissue. The film or sheet can be transparent or opaque. Generally, dehydrated compositions which are dehydrated by lyophilization (freeze-dried) are thicker and opaquer than dehydrated compositions which are dehydrated by evaporative air-drying. Although non-limiting, evaporative air-dried compositions could be considered films whereas lyophilized compositions could be considered sheets.

### C. Excipients and Additional Ingredients

The compositions disclosed herein can further comprise excipients, functional ingredients, and additional ingredients. Non-limiting examples of such ingredients include active pharmaceutical ingredients (APIs), absorbents, antimicrobial agents, antioxidants, antibiofilm agents, binders, buffering agents (e.g., Tris buffer solutions and PBS), bulking agents, chelating agents, colorants, debriding agents, dyes, biocides, deodorant agents, emulsion stabilizers, film formers, fragrance ingredients, humectants, gellants (e.g., cellulose ethers, microcrystalline cellulose, acrylic polymers, alginates, gums, organoclays), lytic agents, enzymes, proteolytic enzymes, opacifying agents, oxidizing agents, pH adjusters, plasticizers, preservatives (e.g., methylparaben, propylparaben, benzyl alcohol), reducing agents, emollients, humectants, hydrophilic polyols, hydrophilic polymeric polyols, polyethylene glycols, moisturizers, surfactants, emulsifying agents, cleansing agents, foaming agents, hydrotopes, solvents, suspending agents, rheology modifiers, viscosity control agents, viscosity increasing agents (e.g., thickeners), vulnerary agents, and propellants. In some embodiments, the composition does not include or contain an enzyme, a proteolytic enzyme, an antibiofilm agent, and/or a debriding agent. In some embodiments, the composition does not include or contain an active pharmaceutical ingredient.

### II. Methods of Use

The compositions disclosed herein are useful for the treatment of wounds of a subject by applying the compositions to or on the wound. A wound can include a disruption of the structure and function of tissue. Non-limiting examples of wounds include: internal organ wounds; mucous membrane wounds; vascular tissue wounds; soft tissue wounds including ligaments, tendons, and cartilage; bone wounds; and dermal wounds. In some embodiments, the wound is a dermal wound. In some embodiments, the composition is applied topically to a dermal wound. In some embodiments, the composition after application is in direct contact with at least a portion of the wound surface.

A dermal wound can involve the disruption of the skin and associated soft tissue architecture. Dermal wounds can be partial or full thickness wounds. They can also be acute wounds, chronic wounds, or burns, which can be acute or chronic. Non-limiting examples of a burn wound include a superficial (first degree) burn, a partial thickness (second degree) burn, a full thickness (third degree) burn, or a radiation burn. Non-limiting examples of a chronic wound include a dermal ulcer, a diabetic ulcer, a diabetic foot ulcer, a venous ulcer, a venous leg ulcer, an arterial ulcer, an arterial leg ulcer, a decubitus ulcer, a stasis ulcer, an ischemic ulcer, a vascular ulcer, a pressure ulcer (stage I-IV), a podiatric wound, a draining wound, a tunneling wound, or an undermining wound. Non-limiting examples of an acute wound include a trauma wound, a laceration, an abrasion, a skin tear, a skin lesion, a blister, a surgical incision, a donor skin site, a skin graft, a laser surgery wound, a Mohs surgery wound, or a dehisced wound. In some embodiments, the dermal wound includes necrotic tissue. In other embodiments, the dermal wound does not contain necrotic tissue.

The compositions of the present invention can be applied to the wound periodically, for example, daily. A therapeutic regiment could be followed to include periodic dressing changes with wound cleansing and application of fresh composition between changes until the wound is healed. The compositions can be applied in conjunction with the application of other wound dressings including but not limited to gauze bandages, sponge wound dressings, foam wound dressings (e.g., Allevyn™ foam dressing), antimicrobial wound dressings, ECM based wound dressings, placental tissue wound dressings, wound debriding dressings, calcium alginate dressings, hydrogels, and wound dressings with vulnerary agents. For example, after application of the composition, the wound may be covered with another wound dressing. The composition can be applied before or after the application of another wound dressing. In some embodiments, the composition comprises lyophilized decidua tissue powder and is applied to the wound before the application of another wound dressing, and in other embodiments, the composition is applied after the application of the other wound dressing. Dehydrated films and sheet compositions can be applied to the wound dry or moistened with an aqueous medium such as saline solution before or after application.

The compositions of the present invention can allow for the cellular migration, vascular ingrowth, and/or the formation of granulation tissues when the compositions are applied to a wound or damaged tissue, which can be demonstrated in a non-limiting way by in vitro by the cellular attachment and proliferation of mesenchymal cells in culture with the compositions. Thus, use of the compositions support and facilitate the wound healing process.

### III. Packaging

The compositions of the present invention can be packaged in any package configuration suitable, for example, for use in storing, shipping, and/or using the compositions of the present invention, topical products. Non-limiting examples of packaging configurations can include containers such as plastic packages, foil packages, pouches, packets, boxes, etc. In in certain aspects where the composition is flowable (e.g., in liquid or hydrogel form), the compositions bottles, jars, bottles with pumps, toddles, tubes (aluminum, plastic or laminated), jars, non-aerosol pump sprayers, and/or aerosol containers could be used. The packages can be configured for single-dose or multiple-dose administration.

Containers such as kits that have multiple compartments can also be used. For instance, a composition of the present invention can be in the form of a film or sheet and be placed in one compartment. A second compartment can include a composition that can include, for example, water or other aqueous solution. This can allow the film or sheet to be mixed with the second composition to form a hydrogel. The kit could also include a mixer (e.g., spoon or rod or paddle, etc.) to mix the two compositions and/or an applicator (e.g., spoon or rod or paddle, etc.) to apply the hydrogel to a wound or tissue such as damaged tissue. Kits can also include 3, 4, 5, or more additional compartments or containers.

Packaging can also include instructions relating to the compositions of the present invention. Instructions can include an explanation of how to apply, use, and maintain the products or compositions.

### EXAMPLES

### Example 1—Lyophilized Decidua Tissue Powder

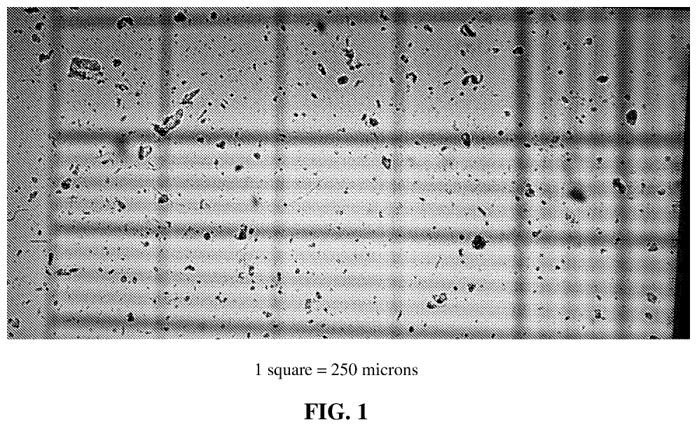

For preparation of lyophilized decidua tissue powder, the unprocessed decidua tissue was weighed and transferred to a Retsch GM200 blender with PBS at an equal weight as the tissue. Then the blender was run at 7000 rpm for 1.5 minutes to produce a homogenized tissue. The tissue was collected by centrifugation. ACK RBC Lysing Buffer was added to the tissue at a 3:1 ratio in volume and remained in contact with the tissue for 30 minutes. After the lysing treatment, the tissue was washed twice with PBS by centrifugation. Then, the tissue was dehydrated by freeze-drying in a lyophilizer. The lyophilized tissue was then powdered using a MACS Octo Dissociator forming a course white to off-white powder. The particle size of the powder was about 90% less than 250 microns as determined microscopically (see FIG. 1).

### Example 2—Dehydrated Film and Sheet Compositions

For all lots, the film and sheet formulations shown in Tables 1, 2, 3, 4 and 5 below were prepared by first preparing a hydrogel (hydrous state) including lyophilized decidua tissue powder, then dehydrating the hydrogel by evaporative air-drying at room temperature and/or lyophilizing to form the film or sheet (dehydrated state). The quantitative formulas show the amount in grams of the components of the hydrogel in the hydrous state prior to dehydration and show the calculated % w/w of the components of the resultant films or sheets in the dehydrated state after dehydration for each lot. For all lots, the HPC (hydroxypropyl cellulose) used was Product number HY121, 4,000-6,500 cP, NF from Spectrum and the HEC (hydroxyethyl cellulose) used was NATROSOL™ 250 HX Pharm from Ashland. For all lots, the films and sheets were prepared by first making a hydrogel by the following process: the HEC, HPC and PEG-600 were added to DI water at RT and mixed until a clear hydrogel was formed. The dehydrated decidua tissue powder was wetted with a small amount of normal phosphate buffer saline solution (PBS) at approximately 20 parts PBS to 1 part decidua powder on a weight basis to form a slurry and then homogenized using a Tissue Tearor™ small homogenizer. Then the homogenized slurry was added to the hydrogel and mixed until uniform. Each lot of the hydrogels were split into samples for dehydration by evaporative air-drying and/or samples for dehydration by lyophilizing to form the resultant film and sheet compositions in the dehydrated state.

TABLE 1

| | Lot | | | | | | | |
|---|---|---|---|---|---|---|---|---|
| | 0055-0114L01 | | 0055-0127L01 | | 0055-0225L01 | | 0055-0331L01 | |
| | gm, | % w/w, | gm, | % w/w, | gm, | % w/w, | gm, | % w/w, |
| | hydrous | dehydrated | hydrous | dehydrated | hydrous | dehydrated | hydrous | dehydrated |
| Component | state | state | state | state | state | state | state | state |
| HPC | 0.48 | 33.48 | 0.71 | 31.50 | 0.54 | 26.52 | 0.55 | 21.27 |
| DI Water | 146.30 | — | 146.30 | — | 146.30 | — | 146.30 | — |
| HEC | 0.57 | 39.15 | 0.83 | 36.49 | 0.63 | 30.79 | 0.64 | 24.74 |
| Lyophilized | 0.07 | 4.62 | 0.24 | 10.78 | 0.51 | 24.82 | 1.02 | 39.45 |
| Decidua | | | | | | | | |
| Tissue | | | | | | | | |
| Powder | | | | | | | | |
| PEG-600 | 0.33 | 22.76 | 0.48 | 21.22 | 0.37 | 17.87 | 0.38 | 14.54 |
| Total | 147.75 | 100.00 | 148.56 | 100.00 | 148.34 | 100.00 | 148.89 | 100.00 |

TABLE 2

| | Lot | | | | | | | |
|---|---|---|---|---|---|---|---|---|
| | 0055-0519L01 | | 0055-0519L02 | | 0055-0519L03 | | 0055-0519L04 | |
| | gm, | % w/w, | gm, | % w/w, | gm, | % w/w, | gm, | % w/w, |
| | hydrous | dehydrated | hydrous | dehydrated | hydrous | dehydrated | hydrous | dehydrated |
| Component | state | state | state | state | state | state | state | state |
| HPC | 1.47 | 33.40 | 1.45 | 33.36 | 1.45 | 33.32 | 1.38 | 31.63 |
| DI Water | 102.00 | — | 101.50 | — | 101.50 | — | 101.00 | — |
| HEC | 1.72 | 39.02 | 1.70 | 38.97 | 1.70 | 38.98 | 1.61 | 36.95 |
| Lyophilized | 0.21 | 4.82 | 0.21 | 4.80 | 0.21 | 4.82 | 0.42 | 9.74 |
| Decidua | | | | | | | | |
| Tissue | | | | | | | | |
| Powder | | | | | | | | |
| PEG-600 | 1.00 | 22.75 | 1.00 | 22.87 | 1.00 | 22.88 | 0.94 | 21.68 |
| Total | 106.40 | 100.00 | 105.85 | 100.00 | 105.86 | 100.00 | 105.35 | 100.00 |

TABLE 3

| | Lot | | | | | | | |
|---|---|---|---|---|---|---|---|---|
| | 0055-0519L05 | | 0055-0519L06 | | 0055-0520L01 | | 0055-0520L02 | |
| | gm, | % w/w, | gm, | % w/w, | gm, | % w/w, | gm, | % w/w, |
| | hydrous | dehydrated | hydrous | dehydrated | hydrous | dehydrated | hydrous | dehydrated |
| Component | state | state | state | state | state | state | state | state |
| HPC | 1.39 | 31.44 | 1.39 | 31.44 | 1.10 | 26.26 | 1.10 | 26.35 |
| DI Water | 105.00 | — | 105.00 | — | 104.00 | — | 104.00 | — |
| HEC | 1.62 | 36.77 | 1.62 | 36.77 | 1.29 | 30.68 | 1.28 | 30.69 |
| Lyophilized | 0.44 | 9.99 | 0.44 | 9.99 | 1.05 | 25.01 | 1.04 | 24.92 |
| Decidua | | | | | | | | |
| Tissue | | | | | | | | |
| Powder | | | | | | | | |
| PEG-600 | 0.96 | 21.79 | 0.96 | 21.79 | 0.76 | 18.01 | 0.75 | 18.04 |
| Total | 109.41 | 99.99 | 109.41 | 99.99 | 108.20 | 99.96 | 108.17 | 100.00 |

TABLE 4

| | Lot | | | | | | | |
|---|---|---|---|---|---|---|---|---|
| | 0055-0520L03 | | 0055-0520L04 | | 0055-0520L05 | | 0055-0520L06 | |
| | gm, | % w/w, | gm, | % w/w, | gm, | % w/w, | gm, | % w/w, |
| | hydrous | dehydrated | hydrous | dehydrated | hydrous | dehydrated | hydrous | dehydrated |
| Component | state | state | state | state | state | state | state | state |
| HPC | 1.10 | 26.35 | 0.88 | 20.48 | 0.88 | 20.30 | 0.88 | 20.38 |
| DI Water | 104.00 | 0.00 | 104.00 | 0.00 | 100.00 | 0.00 | 100.00 | 0.00 |
| HEC | 1.28 | 30.69 | 1.03 | 23.91 | 1.08 | 24.86 | 1.08 | 24.90 |
| Lyophilized | 1.04 | 24.92 | 1.79 | 41.56 | 1.75 | 40.22 | 1.73 | 40.07 |
| Decidua | | | | | | | | |
| Tissue | | | | | | | | |
| Powder | | | | | | | | |
| PEG-600 | 0.75 | 18.04 | 0.61 | 14.05 | 0.63 | 14.61 | 0.63 | 14.61 |
| Total | 108.17 | 100.00 | 108.31 | 100.00 | 104.34 | 99.99 | 104.32 | 99.96 |

TABLE 5

| | Lot | | | | | |
|---|---|---|---|---|---|---|
| | 0055-1102L01 | | 0055-1102L02 | | 0055-1102L03 | |
| | gm, | % w/w, | gm, | % w/w, | gm, | % w/w, |
| | hydrous | dehydrated | hydrous | dehydrated | hydrous | dehydrated |
| Component | state | state | state | state | state | state |
| HPC | 1.30 | 26.10 | 1.31 | 26.12 | 1.31 | 25.97 |
| DI Water | 100 | 0 | 100 | 0 | 100 | 0 |
| HEC | 1.52 | 30.51 | 1.53 | 30.54 | 1.55 | 30.56 |
| Lyophilized | 1.25 | 25.09 | 1.25 | 25.00 | 1.27 | 25.12 |
| Decidua | | | | | | |
| Tissue | | | | | | |
| Powder | | | | | | |
| PEG-600 | 0.91 | 18.30 | 0.92 | 18.33 | 0.93 | 18.34 |
| Total | 104.98 | 100.00 | 105.01 | 100.00 | 105.06 | 99.99 |

For the lots in Table 5, the particle size of the lyophilized decidua tissue powder was 425-1000 microns for the powder used in lot 0055-1102L01; was 216-425 microns for the powder used in lot 0055-1102L02; and was <216 microns for the powder used in lot 0055-1102L03. The powder used in the lots was obtained using 1000-micron, 425-micron, and 216-micron size stacked sieves by adding the lyophilized decidua tissue powder to the 1000 micron sieve, shaking, and then collecting the powder remaining on the 425 micron sieve (425-1000 microns), the 216 micron sieve (216-425 microns), and the powder that passed through the 216 micron sieve (<216 microns).

The evaporative air-dried samples were dehydrated by the following process: approximately 2-3 grams of the hydrogels were poured into 16-well plates used as molds. The molds were covered for 1 hour to ensure the equal spreading of the gel in the mold. The molds were left uncovered at RT under gravity convection conditions to dry for up to 120 hours.

The lyophilized samples were dehydrated by the following process: approximately 2-3 grams of the hydrogels were poured into 16-well plates used as molds. The molds were covered for 1 hour to ensure the equal spreading of the gel in the mold. The molds were placed in a freezer at −80° C. for at least 1 hour to ensure freezing. Once frozen, the molds were placed in a lyophilizer and dried for at least 1 week.

### Results:

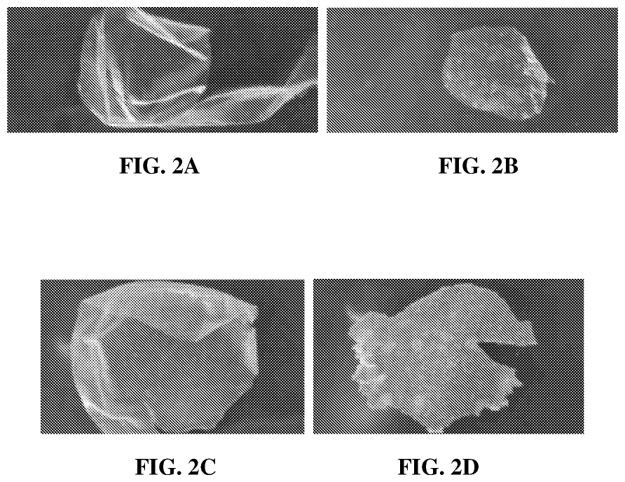

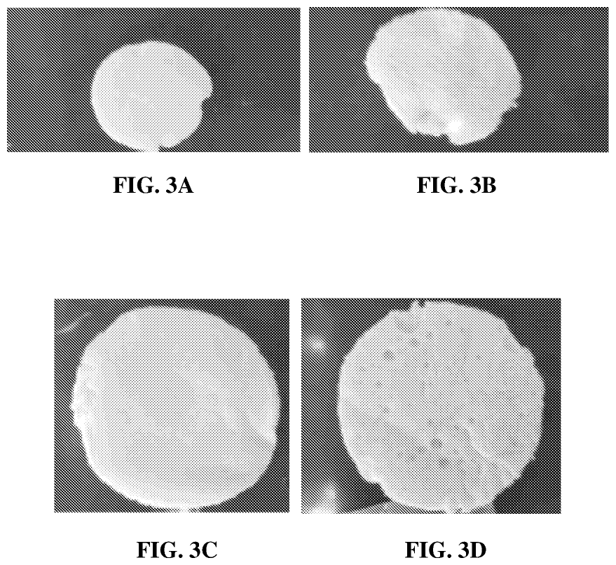

The resulting evaporative air-dried films, also known as xerogels, in lots from Tables 1, 2, 3, & 4 were thin and transparent with an average thickness of 0.8 mm (see FIGS. 2A, 2B, 2C, & 2D). The resulting lyophilized sheets in lots from Tables 1, 2, 3, & 4 were spongy and opaque with an average thickness of 5.1 mm (see FIGS. 3A, 3B, 3C, & 3D). The thickness of the resulting lyophilized sheets in lots from Table 5 was 2 mm for all lots. The water content (moisture content) of the lyophilized sheets from lots in Table 5 was determined by placing the sheets in a 65° C. oven for 3 minutes at atmospheric pressure and reading the weight before and after incubation to determine the weight loss. The water content for lot 005-1102L01 was 0.745% w/w; the water content for lot 005-1102L02 was 1.07% w/w; and the water content for lot 005-1102L03 was 2.59% w/w.

### Example 3—Cell Culture of MSCs with Lyophilized Decidua Tissue Powder and Dehydrated Film and Sheet Compositions

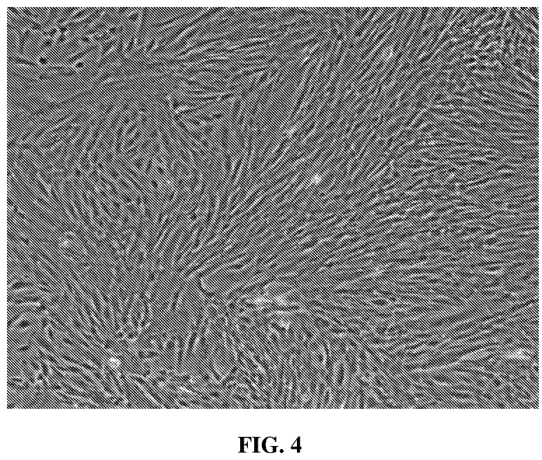

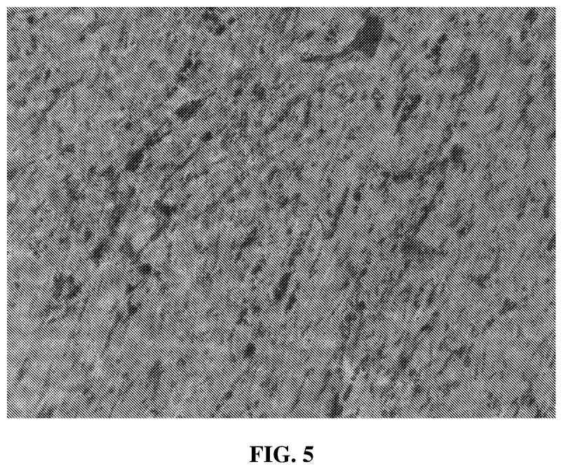

Lyophilized Decidua Tissue Powder: human mesenchymal cells (hMSCs) were cultured with lyophilized decidua tissue powder from Example 1 in hMSCs culture media, i.e., DMEM+10% FBS at 37° C., in petri dishes. The photomicrograph in FIG. 4 of the cell culture plate after culturing indicates that the lyophilized decidua tissue powder induces cell growth, migration, and proliferation of hMSCs. An additional study shows the differentiation of the proliferated hMSCs to adipocytes as seen in the photomicrograph in FIG. 5.

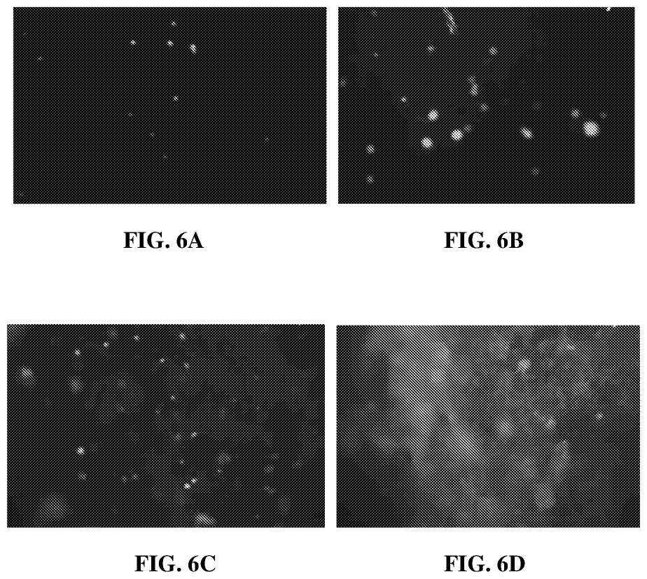

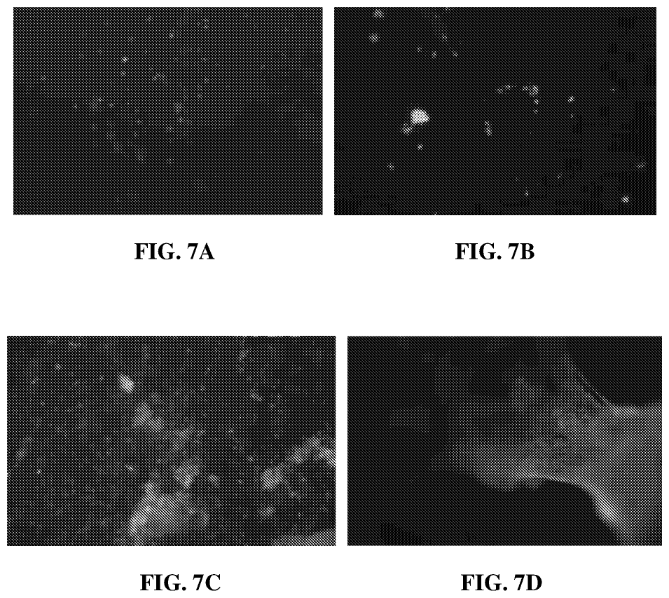

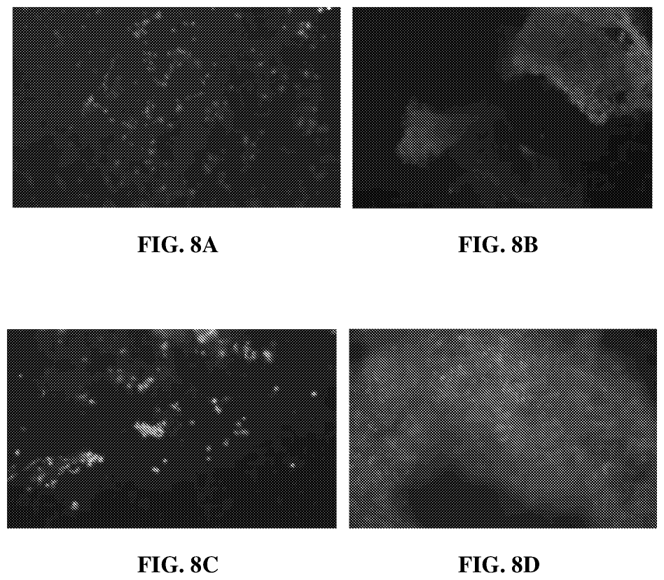

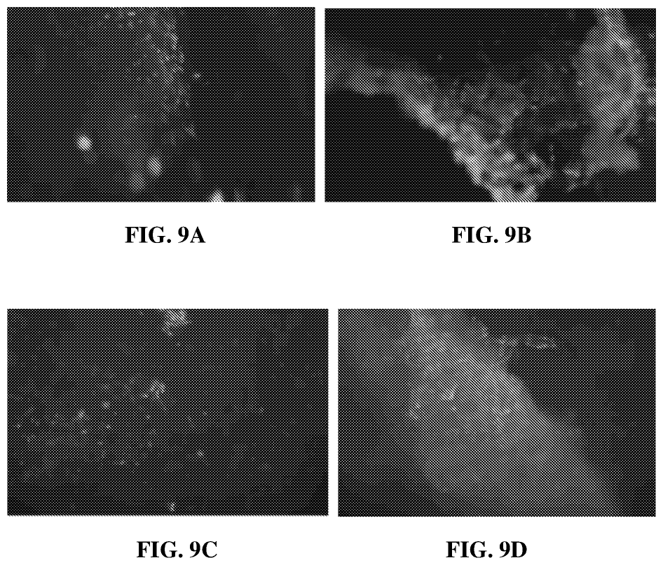

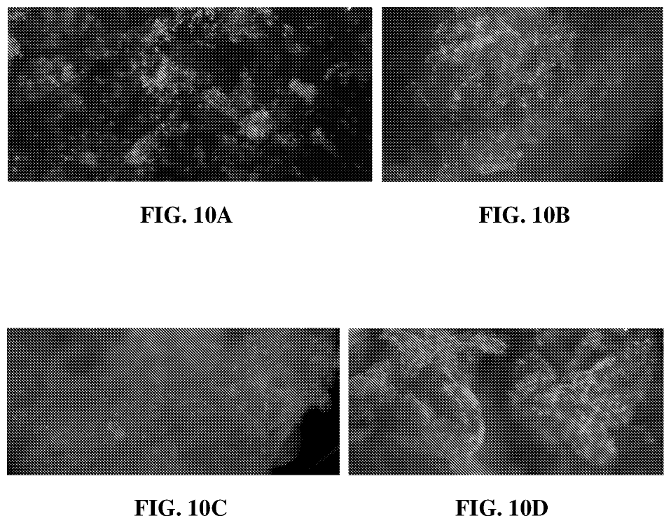

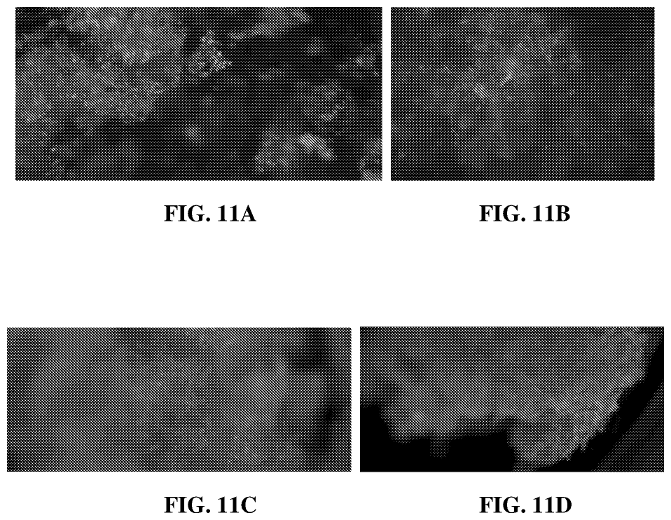

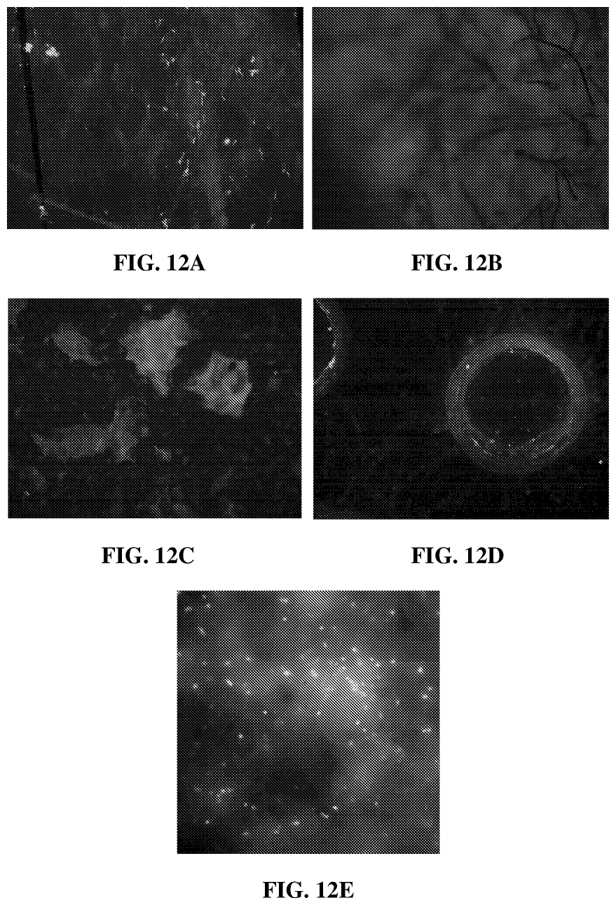

Dehydrated Film and Sheet Compositions: human mesenchymal cells (hMSCs) were cultured with the films dehydrated by evaporative air-drying and the sheets dehydrated by lyophilization from Example 2 in hMSCs culture medium, i.e., DMEM+10% FBS, at 37° C. in 12-well cell culture plates. 0.5 M cells were added per well in 3 ml of medium and were cultured for several days up to 27 days. Using the ThermoFisher LIVE/DEAD® Viability/Cytotoxicity Kit for Mammalian Cells Protocol and following the protocol outlined in the ThermoFisher webpage https://www.thermofisher.com/us/en/home/references/protocols/cell-and-tissue-analysis/protocols/live-dead-viability-cytotoxicity-kit-for-mammalian-cells.html herein incorporated by reference, live/dead cell stains were taken at various days after initiating the culture and photomicrographs were taken of the stains at those times (see FIGS. 6A-D, 7A-D, 8A-D. 9A-D, 10A-D, & 11A-D). As seen in the photomicrographs, both the films dehydrated by evaporative air-drying and the sheets dehydrated by lyophilization induce cell growth, migration, and proliferation of hMSCs in culture. A variety of commercially available wound dressings were also cultured with hMSCs for comparison purposes. Live/dead cell stains were taken at 7 days after initiating the culture and photomicrographs were taken at that time (see FIGS. 12A-D). As seen in the photomicrographs, these commercially available wound dressing products showed far less cell growth and proliferation than the dehydrated film and sheet compositions. Thus, the results indicate that the dehydrated film and sheet compositions show cell growth, migration, and proliferation of hMSCs in culture and show superior properties with respect to cell growth, migration, and proliferation of hMSCs in culture than various commercially available wound dressings.

### Example 4—Rehydration of Dehydrated Film and Sheet Compositions Forming a Hydrogel and Viscosity Measurements of the Hydrogel

The dehydrated lyophilized compositions of the lots from Table 5 above were rehydrated by adding about 1 part by weight of the lyophilized dehydrated sheet into about 20 parts by volume of normal saline solution to form a hydrogel. The viscosity measurement was conducted on a sample of the resultant hydrogel using a Brookfield viscometer Model RV-DV2T cone & plate viscometer using cone spindle CP-52 at 0.5 RPM. The measurement was run at RT for 1 min. The viscosity results are reported in centipoise (cps). The viscosity value for lot 0055-1102L01 was 12,900 cps; the viscosity value for lot 0055-1102L01 was 13,250 cps; and the viscosity value for lot 0055-1102L03 was 12,870 cps.

### Example 5—In Vivo Animal Studies

Purpose: To evaluate local tissue response and wound closure from treatment with dehydrated decidua compositions on a porcine animal model.

Porcine Animal Model: Full-thickness excisional wounds, 3 cm×3 cm, with 3 to 6 mm depth (dependent on pig) will be generated on the pigs. The dehydrated decidua compositions will be placed on the wounds as treatment. Controls, such as a predicate device, will also be set up.

In-life Timepoints: Weekly wound images and wound measurements will be conducted with documentation of any other macroscopic observations.

Histology Timepoints: At a single timepoint at 28 days, H&E and Masson's Trichrome of a single section through the middle of the wound will be conducted with pathology scoring. Additional immunohistochemistry may be added later.

Endpoint: At the end of the study, the histology will be evaluated and the degree of wound closure on the animals will be determined for the dehydrated decidua tissue compositions and the controls.

1. A composition comprising dehydrated decidua tissue.

2. The composition of claim 1, wherein the dehydrated decidua tissue comprises decidua basalis tissue, decidua capsularis tissue, or decidua parietalis tissue, or any combination thereof.

3. The composition of claim 1, wherein the dehydrated decidua tissue is non-viable.

4. The composition of claim 1, wherein the dehydrated decidua tissue is a powder.

5. The composition of claim 1, wherein the dehydrated decidua tissue is lyophilized.

6. The composition of claim 1, wherein the water content of the dehydrated decidua tissue is less than 5% w/w.

7. The composition of claim 1 further comprising a pharmaceutically acceptable carrier.

8. The composition of claim 7, wherein the pharmaceutically acceptable carrier comprises at least 50% w/w water.

9. The composition of claim 1, wherein the pharmaceutically acceptable carrier comprises one or more cellulose ethers.

10. The composition of claim 9, wherein the cellulose ether is a non-ionic cellulose ether, wherein the non-ionic cellulose ether is selected from hydroxyethyl cellulose (HEC) or hydroxypropyl cellulose (HPC), or mixtures thereof.

11. (canceled)

12. The composition of claim 1, wherein the pharmaceutical acceptable carrier further comprises one or more plasticizers, wherein the one or more plasticizers is a hydrophilic polyol.

13. (canceled)

14. The composition of claim 12, wherein the hydrophilic polyol is a polyethylene glycol.

15. (canceled)

16. The composition of claim 14, wherein the one or more polyethylene glycols comprises polyethylene glycol 600.

17. The composition of claim 1, wherein the composition is dehydrated.

18. (canceled)

19. (canceled)

20. (canceled)

21. The composition of claim 17, wherein the water content of the dehydrated composition is less than 5% w/w.

22. The composition of claim 17, wherein the dehydrated composition is a sheet or film having a thickness of from about 0.1 mm to about 25 mm.

23. (canceled)

24. The composition of claim 17, wherein the concentration of decidua tissue is from about 1% to about 75% w/w of the dehydrated composition.

25. The composition of claim 17, wherein the dehydrated composition is capable of forming a hydrogel when the dehydrated composition is contacted with water or other aqueous medium.

26. The composition of claim 17, wherein the dehydrated composition is opaque.

27. The composition of claim 17, wherein the dehydrated composition is transparent.

28. A method of treating a wound in a subject, the method comprising applying the composition of claim 1 to the wound.

29-37. (canceled)

38. A method of making a dehydrated composition comprising dehydrated decidua tissue, the method comprising:
(a) providing dehydrated decidua tissue;
(b) providing an aqueous based pharmaceutically acceptable carrier;
(c) combining (a) and (b) and mixing until uniform; and
(d) dehydrating the resultant mixture, thereby forming the dehydrated composition;
wherein the aqueous based carrier has a water content of at least 50% w/w, wherein the dehydrated composition is a film or sheet, and wherein the film or sheet has a water content of less than 5% w/w or less than 3% w/w.

39-43. (canceled)
